(19) United States
(12) Patent Application Publication
(10) Pub. No.: US 2026/0335097 A1
(43) Pub. Date: Oct. 29, 2026

(54) Total PEEK Wedge Implant System

(71) Applicants: Thomas Zink, San Antonio, TX (US); Natalia Reyes, San Antonio, TX (US); James Saunders, San Antonio, TX (US)

(72) Inventors: Thomas Zink, San Antonio, TX (US); Natalia Reyes, San Antonio, TX (US); James Saunders, San Antonio, TX (US)

(21) Appl. No.: 19/410,834

(22) Filed: Dec. 5, 2025

Related U.S. Application Data
(60) Provisional application No. 63/794,735, filed on Apr. 25, 2025.

(51) Int. Cl. A61F 2/42; A61B 17/00; A61B 17/68; A61B 17/86; A61F 2/30; A61L 27/46

(52) U.S. Cl. CPC A61F 2/4225; A61B 17/8615; A61B 17/8625; A61F 2/30749; A61F 2/30942; A61F 2/4202; A61L 27/46; A61B 2017/00526; A61B 2017/00933; A61B 2017/681; A61B 2017/8655; A61F 2002/30266; A61F 2002/30622; A61F 2002/30841; A61F 2002/30985; A61F 2002/4223; A61L 2430/02

(57) ABSTRACT

A total PEEK wedge implant system is disclosed. The wedge implant of the system is designed for bi-planar correction, specifically, one plane of correction is in the transverse plane to bring the toe back into alignment; and a second plane of correction is in the sagittal plane to allow for the proper gate of the foot. A pair of pins are used to fixate the wedge implant into place. Both the wedge implant and the pair of pins are constructed of fusion-grade PEEK (polyether ether ketone).

### CROSS-REFERENCE TO RELATED APPLICATION

The present application claims priority to, and the benefit of, U.S. Provisional Application No. 63/794,735, which was filed on Apr. 25, 2025 and is incorporated herein by reference in its entirety.

### FIELD OF THE INVENTION

The present invention relates generally to a medical device for use in the field of podiatry. More particularly, the present invention relates to an implant system used to correct and reconstruct the arch and location of bones in the foot. The disclosed implant system is used to push bones and joints in directions that correct deformity or degeneration of the foot. Accordingly, the present disclosure makes specific reference thereto. Nonetheless, it is to be appreciated that aspects of the present invention are also equally applicable to other like applications, devices and methods of manufacture.

### BACKGROUND

Wedges have long been used in the foot and ankle orthopedic space to correct and reconstruct the arch and location of bones in the foot. Generally, the wedges are used to push bones and joints in directions that correct deformity or degeneration of the foot. Typical reconstruction procedures include the Lapidus procedure, which is commonly used to correct a hallux valgus (bunion) deformity, which is a lateral deviation of the great toe, with subsequent hypermobility (or laxity). Generally, a bunion is caused by a malalignment of the first metatarsal and the proximal phalanx of the hallux.

Another reconstruction procedure includes an Evans osteotomy, which is an osteotomy of the calcaneas or the heel bone. An osteotomy is a surgical operation whereby a bone is cut to shorten, lengthen, or change its alignment. The Evans osteotomy is a lateral based opening wedge osteotomy that effectively lengthens the lateral column thus reducing forefoot abduction and transverse plane deformity. It offers triplanar correction of the symptomatic flexible flatfoot by adducting and plantarflexing the forefoot and supinating the subtalar joint.

Another way to surgically correct a patient's flatfoot deformity is the Cotton osteotomy. The Cotton osteotomy is a medial opening wedge osteotomy that is performed in the first cuneiform bone of the foot. Like the Evans, the Cotton osteotomy is a surgically corrective osteotomy to assist in the correction of the flatfoot deformity. Like the Evans, the Cotton osteotomy is an opening wedge osteotomy where the first cuneiform bone is “opened” by the osteotomy cut of the bone to obtain a certain amount of correction.

The Lapidus procedure is also commonly used to repair failed surgeries. Typically, a wedge of bone is removed in a biplanar direction at the distal end of the cuneiform, which will provide correction of the deformity and typically results in shortening of the great toe. The result of this shortening is a shift in weight distribution to the second ray, which can result in metatarsalgia. When the first ray is shortened the function of the patient's sesamoids may also be affected because of the change in weight distribution on the sesamoids.

Previously, wedges and fixation are normally done with titanium, printed titanium, allograft bone, or standard PEEK. Further, any fixation is accomplished with the addition of titanium or stainless steel plates with titanium or stainless steel screws. However, metal allergies are on the rise and most patients do not want metal in their body long term. Metal also makes post-operative imaging problematic, it can produce artifacts in CT imaging and it is opaque on x-ray not allowing the surgeon to see bone fusion. However, the use of (polyether-ether-ketone) PEEK in such implants does not allow for osteo-conduction. Therefore, fusion-grade PEEK has been formulated to allow for such osteo-conduction. Accordingly, in view of the deficiencies of the current implants, it would be desirable to develop implants and pins using advanced materials, such as fusion-grade PEEK materials.

Accordingly, it would be desirable to provide biplanar correction wedges additively manufactured using fusion-grade PEEK. Further, it would also be desirable to utilize machined, fusion-grade PEEK pins to secure the wedges into place.

The subject matter disclosed and claimed herein, in one embodiment thereof, comprises a total PEEK wedge implant system. The wedge implant is designed for bi-planar correction, specifically, one plane of correction is located in the transverse plane to bring the toe back into alignment; and a second plane of correction is located in the sagittal plane to allow for the proper gate of the foot. The total PEEK wedge implant system comprises an anatomically shaped fusion-grade PEEK (polyether ether ketone) wedge implant used to maintain length and optimize positioning of the first ray in both transverse and sagittal planes and a pair of fusion-grade PEEK pins used to fixate the wedge implant into place. The wedge implant is provided in various length maintaining heights and angle variations for correction of the first ray in both transverse and sagittal planes. The pair of pins are impacted into place instead of screwed into place. Typically, both the wedge implant and the pair of pins are manufactured of fusion-grade PEEK.

In this manner, the total PEEK wedge implant system of the present invention accomplishes all of the forgoing objectives and provides users with an implant and pair of pins that are manufactured of fusion-grade PEEK so they are inert, and do not resorb into the surrounding bone. The wedge implant provides bi-planar correction and is anatomically shaped. The pair of pins are impacted into place to add to the overall fixation of the wedge implant during use.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one embodiment thereof, comprises a total PEEK wedge implant system. The system is designed for biplanar correction of the foot. The system comprises an anatomically shaped fusion-grade PEEK (polyether ether ketone) wedge implant used to maintain length and optimize positioning of the first ray in both transverse and sagittal planes and a pair of fusion-grade PEEK pins that allow for overall fixation.

The wedge implant is provided in various length maintaining heights and angle variations for correction of the first ray in both transverse and sagittal planes. The wedge implant is used for aiding or providing internal fixation of calcaneus, tarsal, and metatarsal bones of the foot in cases such as, but not limited to, filling an osteotomy or expanding and correcting an angular deformity in the foot.

In one embodiment, the wedge implant is manufactured of fusion-grade PEEK. Any suitable fusion-grade PEEK can be utilized as is known in the art, but typically the fusion-grade PEEK is PEEK HA (hydroxyapatite) or PEEK composited with calcium-phosphate.

In one embodiment, the wedge implant comprises a generally wedge-shaped body, with the body having a generally rounded top which recedes into a bottom with a smaller radius, a generally planar first side, and a generally planar second side. The first and second sides each slope inwardly from the top to the bottom. However, the dimensions and angles of the wedge implant may be changed as desired in order to accommodate varying anatomies, purposes, and desired outcomes. It should also be appreciated that the wedge implant and/or its principles are applicable to other bones of the body such as, but not limited to, the bones of the hand.

Further, the body of the wedge implant also comprises two bores each of which are dimensioned to receive a pin, such that the head of the pin is held by the body of the wedge implant and a portion of the tip and/or shank of the pin extends from the body for receipt in a foot bone. The first pin bore extends between the top and the first side such that the tip of a pin extends from the first side at an angle thereof. The opening of the first pin bore on the top of the body is situated proximate to and on the edge between the top and the second side creating the angled pin bore. The second pin bore extends between the top and the second side such that the tip of a pin extends from the second side at an angle thereof. The opening of the second pin bore on the top of the body is situated proximate to and on the edge between the top and the first side creating the angled second pin bore. Both pin openings can be designed for different angulation. Further, both pin bore openings on the top of the body are sized and configured to capture and retain the head of a pin. Moreover, both pin bore openings on the top are configured such that the heads of the pins are countersunk relative to the surface of the top.

In another embodiment, the wedge implant body is secure via a pair of pins. The pair of pins are shaped and sized to be positioned through the bores disclosed supra. In position within the wedge implant body, the set of pins are angled such that they extend out through the implant body opening, away from the wedge implant, and contact a foot bone to add to the overall fixation of the wedge implant. Each of the set of pins is manufactured of fusion-grade PEEK. Any suitable fusion-grade PEEK can be utilized as is known in the art, but typically the fusion-grade PEEK is PEEK HA (hydroxyapatite) or PEEK composited with calcium-phosphate. This allows the pins to be impacted into place instead of screwed into place. Additionally, each of the set of the pins includes a hexalobe insertion feature or any other suitable insertion feature, which aids a user in inserting the pin via impacting the pin into the bone or removing the pin or pins, as needed for revisions. Further, each of the set of the pins includes a plurality of anti-migration ribs along the exterior shank, to prevent the set of pins from migrating out of the bone. The plurality of anti-migration ribs are cut into the exterior of the pins along their length, at predetermined intervals.

In another embodiment, the wedge implant body comprises an opening that extends from one side to the second side. The opening can be any suitable shape and size as is known in the art, depending on the needs and/or wants of a user. In one embodiment, the opening is used to receive and hold bone graft material. As such, the opening may be considered a bone graft window or simply a graft window. The bone graft material may be of any suitable kind as is known in the art which promotes bone growth. In use, the wedge implant body can be placed in a graft packing block and then filled with graft material.

In yet another embodiment, the opening of the wedge implant body is filled with an osteo-integrative lattice. The osteo-integrative lattice promotes bone growth through the wedge implant during use. Thus, instead of an opening, the osteo-integrative lattice creates a solid construct of the wedge implant. Typically, the wedge implant and osteo-integrative lattice are 3D printed together as one piece to provide the solid construct. In this embodiment, bone graft material can still be pressed into the osteo-integrative lattice before insertion according to a user's needs and/or wants to promote additional bone growth.

In one embodiment, in addition to the bone graft window, the body of the wedge implant comprises features for aiding in and/or promoting fusion between two foot bones or foot bone portions. These features also aid in keeping the body of the wedge implant in place. Particularly, a portion of the one side has teeth, serrations or any other suitable anti-migration features, while a portion of the other side likewise has teeth, serrations or any other suitable anti-migration features. The teeth surround the graft window or at least are present about the top and sides of the graft window. Typically, the upper portions of the sides proximate to the top portions of the sides, preferably, but not necessarily, lack teeth, serrations, or other suitable anti-migration features.

In another embodiment, the body of the wedge implant comprises a threaded bore/insertion feature that is provided for engagement with an insertion instrument and/or drill guide, or other insertion instruments as is known in the art.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the total PEEK wedge implant system of the present invention in accordance with the disclosed architecture;

FIG. 2 illustrates a perspective view of the total PEEK wedge implant system of the present invention showing the lattice for osteointegration in accordance with the disclosed architecture;

FIG. 3 illustrates a side perspective view of the total PEEK wedge implant system of the present invention showing the lattice of the wedge implant in accordance with the disclosed architecture;

FIG. 4 illustrates a side perspective view of the total PEEK wedge implant system of the present invention showing transverse plane correction in accordance with the disclosed architecture;

FIG. 5 illustrates a side perspective view of the total PEEK wedge implant system of the present invention showing sagittal plane correction in accordance with the disclosed architecture;

FIG. 6 illustrates a side perspective view of the total PEEK wedge implant system of the present invention showing a pin for fixating the wedge implant in accordance with the disclosed architecture; and

FIG. 7 illustrates a perspective view of the total PEEK wedge implant system of the present invention showing a pin with insertion feature in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

In this application, the words proximal, distal, anterior or plantar, posterior or dorsal, medial and lateral are defined by their standard usage for indicating a particular part or portion of a bone, or directional terms of reference, according to the relative disposition of the natural bone. For example, “proximal” means the portion of a bone nearest the torso, while “distal” indicates the portion of the bone farthest from the torso. As an example of directional usage of the terms, “anterior” refers to a direction towards the front side of the body, “posterior” refers to a direction towards the back side of the body, “medial” refers to a direction towards the midline of the body and “lateral” refers to a direction towards the sides or away from the midline of the body. Further, specifically in regard to the foot, the term “dorsal” refers to the top of the foot and the term “plantar” refers the bottom of the foot.

Similarly, positions or directions may be used herein with reference to anatomical structures or surfaces. For example, as the current devices, instrumentation and methods are described herein with reference to use with the bones of the foot, the bones of the foot, ankle and lower leg may be used to describe the surfaces, positions, directions or orientations of the devices, instrumentation and methods. Further, the devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the devices, instrumentation and methods, and the aspects, components, features and the like thereof, described herein with respect to the right foot may be mirrored so that they likewise function with the left foot. Further, the devices, instrumentation and methods, and the aspects, components, features and the like thereof, disclosed herein are described with respect to the foot for brevity purposes, but it should be understood that the devices, instrumentation, and methods may be used with other bones of the body having similar structures, for example the upper extremity, and more specifically, with the bones of the wrist, hand, and arm.

As noted above, there is a long felt need in the art for devices, instrumentation, and methods which allow for correction of deformity or degeneration of the foot. Specifically, devices, instrumentation and methods which utilize an anatomically shaped implant used to maintain length and optimize positioning of the first ray in both transverse and sagittal planes and a pair of pins for additional fixation of the wedge implant.

The present invention, in one exemplary embodiment, is a novel total PEEK wedge implant system. The system comprises an anatomically shaped fusion-grade PEEK (polyether ether ketone) wedge implant used to maintain length and optimize positioning of the first ray in both transverse and sagittal planes and a pair of fusion-grade pins which are impacted into place for overall fixation. The wedge implant is provided in various length maintaining thicknesses and angle variations for correction of the first ray in both transverse and sagittal planes. The pair of pins include anti-migration ribs and a hexalobe insertion feature.

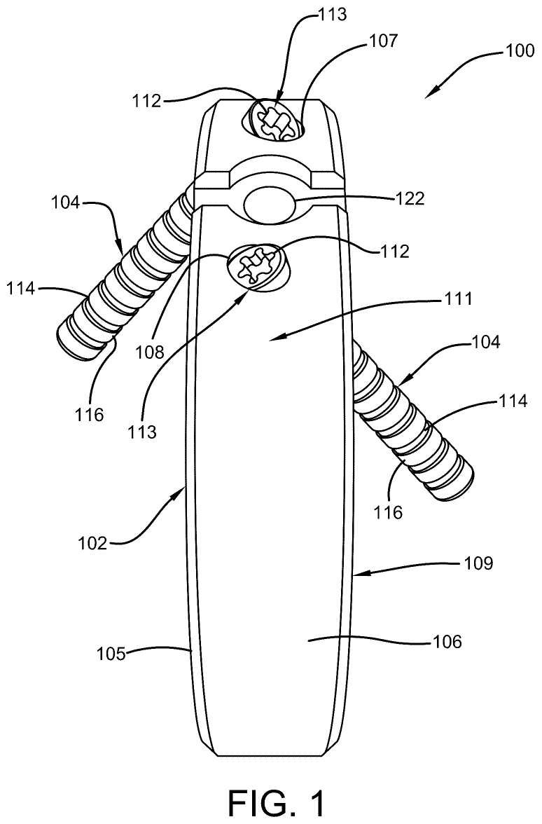

Referring initially to the drawings, FIGS. 1-7 illustrate multiple views of the total PEEK wedge implant system 100 of the present invention. In the present embodiment, the total PEEK wedge implant system 100 is an improved total PEEK wedge implant system 100 that corrects deformity or degeneration of the foot. The system 100 is especially designed to allow any doctor, surgeon, etc., or any other suitable user as is known in the art, to correct and reconstruct the arch and location of bones in the foot. More specifically, the system 100 comprises a wedge implant 102 used to maintain length and optimize positioning of the first ray in both transverse and sagittal planes and a pair of pins 104 that add to the overall fixation of the wedge implant.

Specifically, as illustrated in FIG. 1, one embodiment of the total PEEK wedge implant system 100 is shown. Specifically, the system 100 includes the wedge implant 102 with inserted pins 104. The wedge implant 102 is made to a desired restoration length necessary for the user receiving the implant 102 and has an outer anatomical profile that mimics the anatomical considerations of the bones it is being inserted between. Furthermore, the wedge implant 102 may have heights ranging from about 10 mm to 50 mm, widths ranging from about 10 mm to 40 mm, and thicknesses ranging from about 1 mm to 20 mm, also having angles ranging from about 2 degrees to 30 degrees. However, the dimensions and angles of the wedge implant 102 may be changed as desired in order to accommodate varying anatomies, purposes, and desired outcomes. It should also be appreciated that the wedge implant 102 and/or its principles are applicable to other bones of the body such as, but not limited to, the bones of the hand.

Further, the wedge implant 102 comprises a body 106 comprising at least two bores 108 each of which are dimensioned to receive a pin 104, such that the head 113 of the pin 104 is held by the body 106 of the wedge implant 102 and a portion of the tip and/or shank 116 of the pin 104 extends from the body 106 for receipt in a foot bone. Any suitable number of bores 108 can be utilized as is known in the art, depending on the needs and/or wants of a user. The first pin bore 108 extends between the top 111 and the first side 109 such that the tip of a pin 104 extends from the first side 109 at an angle thereof. The opening of the first pin bore 108 on the top 111 of the body 106 is situated proximate to and on the edge between the top 111 and the first side 109 creating the angled pin bore 108. The second pin bore 107 extends between the top 111 and the second side 105 such that the tip of a pin 104 extends from the second side 105 at an angle thereof. The opening of the second pin bore 107 on the top 111 of the body 106 is situated proximate to and on the edge between the top 111 and the second side 105 creating the angled second pin bore 107. Both pin openings 107, 108 can be designed for different angulation. Further, both pin bore openings 107, 108 on the top 111 of the body 106 are sized and configured to capture and retain the head of a pin 104. Moreover, both pin bore openings 107, 108 on the top 111 are configured such that the heads 113 of the pins 104 are countersunk relative to the surface of the top 111.

Furthermore, the wedge implant body 106 is secure via a pair of pins 104. The pair of pins 104 are shaped and sized to be positioned through the bores 107, 108 disclosed supra. In position within the wedge implant body 106, the set of pins 104 are angled such that they extend out through the implant body opening 110, away from the wedge implant 102, and contact a foot bone to add to the overall fixation of the wedge implant 102. Each of the set of pins 104 is manufactured of fusion-grade PEEK. Any suitable fusion-grade PEEK can be utilized as is known in the art, but typically the fusion-grade PEEK is PEEK HA (hydroxyapatite) or PEEK composited with calcium-phosphate. This allows the pins 104 to be impacted into place instead of screwed into place. Additionally, each of the set of the pins 104 includes a head 113 with a hexalobe insertion feature 112 or any other suitable insertion feature, which aids a user in inserting the pin 104 via impacting the pin 104 into the bone or removing the pin or pins 104, as needed for revisions. Upon insertion, the pin 104 is positioned within the bore 107, 108 of the wedge implant 102 and persuaded into place via a mallet and insertion tamp (not shown). This makes it possible to manufacture the pin 104 out of a radiolucent polymer, such as fusion-grade PEEK, which performs well in compression, especially when being persuaded into place via the mallet. The use of PEEK does not do well with a torsional load, as such a user would not want to twist or torque the pin 104 into the bores 107, 108, as in the prior screw technology. Further, the fusion-grade PEEK material is radiolucent/radiotransparent, which will allow for better post-surgery monitoring using x-ray imaging. Also, PEEK is more workable by drilling or cutting with various surgical instruments.

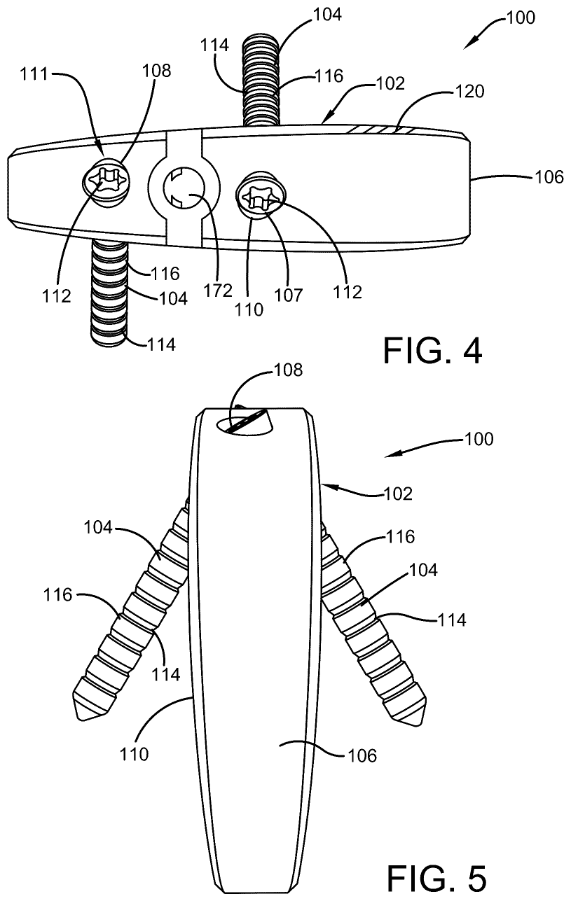

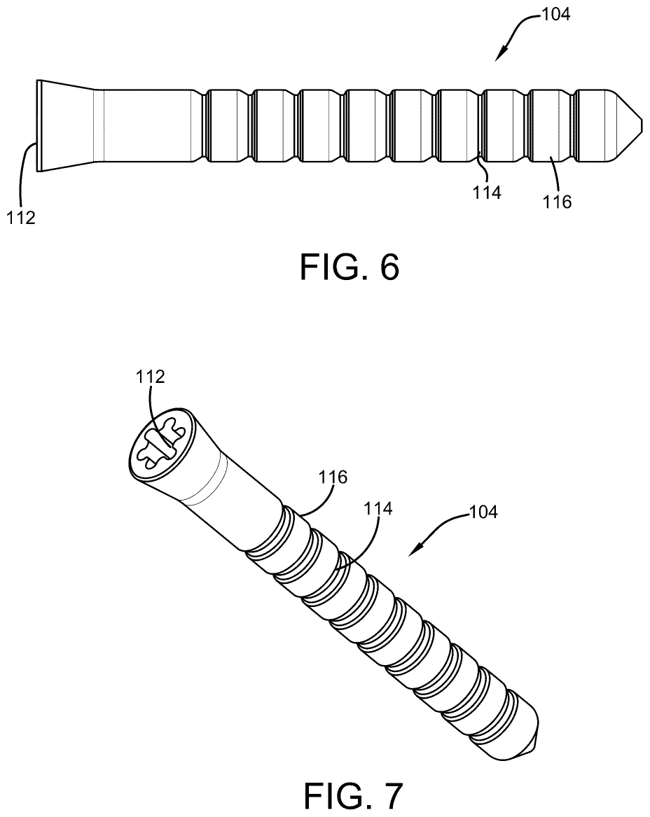

FIG. 4 illustrates a side perspective view of the total PEEK wedge implant system of the present invention showing transverse plane correction in accordance with the disclosed architecture, and FIG. 5 illustrates a side perspective view of the total PEEK wedge implant system of the present invention showing sagittal plane correction in accordance with the disclosed architecture. FIG. 6 illustrates a side perspective view of the total PEEK wedge implant system of the present invention showing a pin for fixating the wedge implant in accordance with the disclosed architecture, and FIG. 7 illustrates a perspective view of the total PEEK wedge implant system of the present invention showing a pin with insertion feature in accordance with the disclosed architecture.

Additionally, each of the set of the pins 104 includes a plurality of anti-migration ribs 114 along the exterior shank 116, to prevent the set of pins 104 from migrating out of the bone. The plurality of anti-migration ribs 114 are cut into the exterior 116 of the pins 104 along their length, at predetermined intervals for optimal fixation. Specifically, the plurality of ribs 114 are cut into the circumference, along the pin's length. The ribs 114 are cut in, such that the surrounding bone enters between the ribs 114 when the pin 104 is inserted. Typically, the ribs 114 are cut in approximately ¼-½ mm radially. Any suitable number of ribs 114 can be utilized as is known in the art. Further, the ribs 114 are configured in a spaced apart manner. Typically, the ribs 114 are spaced apart, such that they have a pitch of approximately 2-3 millimeters to promote boney apposition.

Additionally, the wedge implant body 106 comprises an opening 110 that extends from the first side 109 to the second side 105. The opening 110 can be any suitable shape and size as is known in the art, depending on the needs and/or wants of a user. In one embodiment, the opening 110 is used to receive and hold bone graft material (not shown). As such, the opening 110 may be considered a bone graft window or simply a graft window. The bone graft material may be of any suitable kind as is known in the art to promote bone growth. In use, the wedge implant body 106 can be placed in a graft packing block and then filled with graft material before implantation.

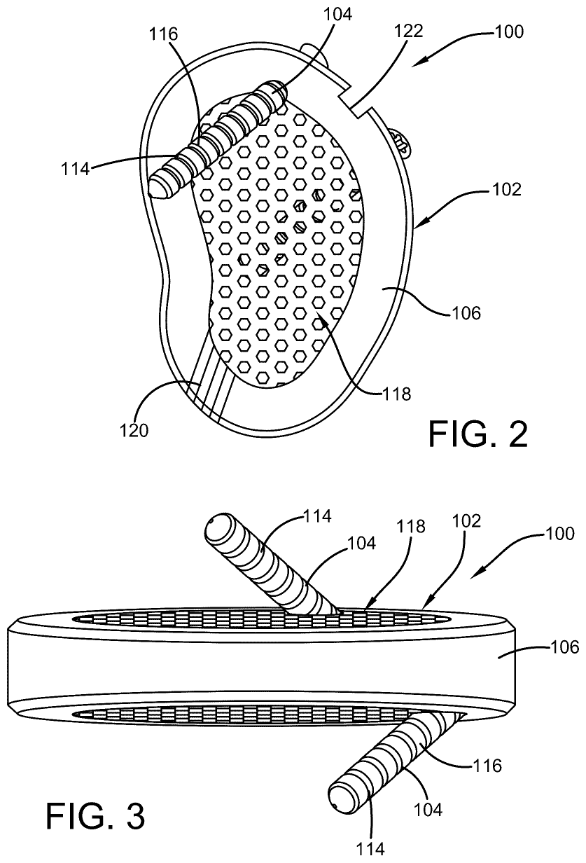

In yet another embodiment, the opening 110 of the wedge implant body 106 is filled with an osteo-integrative lattice 118. FIG. 2 illustrates a perspective view of the total PEEK wedge implant system of the present invention showing the lattice for osteointegration in accordance with the disclosed architecture, and FIG. 3 illustrates a side perspective view of the total PEEK wedge implant system of the present invention showing the lattice of the wedge implant in accordance with the disclosed architecture. The osteo-integrative lattice 118 promotes bone growth through the wedge implant 102 during use and acts as a scaffold for bone. Thus, instead of an opening 110, the osteo-integrative lattice 118 creates a solid construct of the wedge implant 102. Typically, the wedge implant 102 and osteo-integrative lattice 118 are 3D printed together as one piece to provide the solid construct. In this embodiment, bone graft material can still be pressed into the osteo-integrative lattice 118 before insertion according to a user's needs and/or wants to promote additional bone growth.

In addition to the bone graft window 110, the body 106 of the wedge implant 102 comprises features 120 for aiding in and/or promoting fusion between two foot bones or foot bone portions. These features 120 also aid in keeping the body 106 in place. Particularly, a portion of the first side 109 has teeth, serrations or any other suitable anti-migration features 120, while a portion of the second side 105 likewise has teeth, serrations or any other suitable anti-migration features 120. The teeth 120 surround the graft window 110 or at least are present about the top and sides of the graft window 110. Typically, the upper portions of the sides proximate to the top 111 of the sides 105, 109, preferably, but not necessarily, lack teeth, serrations, or other suitable anti-migration features 120.

Further, the body 106 of the wedge implant 102 comprises a threaded bore/insertion feature 122 that is provided for engagement with an insertion instrument and/or drill guide, or other insertion instruments (not shown) as is known in the art for implantation of the implant 102.

In one embodiment, the wedge implant 102 and its pins 104 are manufactured of fusion-grade PEEK. Any suitable fusion-grade PEEK can be utilized as is known in the art, but typically the fusion-grade PEEK is PEEK HA (hydroxyapatite) or PEEK composited with calcium-phosphate. Specifically, the wedge implant 102 is 3D printed or additively manufactured and thus, able to be manufactured in a variety of sizes as well as to be customizable to fit the exact specifications/measurements of the particular user. Typically, the pins 104 are machined due to their size.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “total PEEK wedge implant system”, “wedge system”, and “system” are interchangeable and refer to the total PEEK wedge implant system 100 of the present invention.

Notwithstanding the forgoing, the total PEEK wedge implant system 100 of the present invention can be of any suitable size and configuration as is known in the art without affecting the overall concept of the invention, provided that it accomplishes the above stated objectives. One of ordinary skill in the art will appreciate that the total PEEK wedge implant system 100 as shown in FIGS. 1-7 is for illustrative purposes only, and that many other sizes and shapes of the total PEEK wedge implant system 100 are well within the scope of the present disclosure. Although the dimensions of the total PEEK wedge implant system 100 are important design parameters for user convenience, the total PEEK wedge implant system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A total PEEK wedge implant system comprising:
a wedge implant; and
a pair of pins;
wherein the wedge implant is sized and configured to be utilized to provide a bi-planar correction in a patient; and
further wherein the pair of pins are impacted into place within the wedge implant to add overall fixation to the wedge implant.

2. The total PEEK wedge implant system of claim 1, wherein the bi-planar correction includes correction in a transverse plane to bring a toe back into alignment and correction in a sagittal plane to allow for proper gate of a foot.

3. The total PEEK wedge implant system of claim 2, wherein the wedge implant and the pair of pins are both manufactured of a PEEK HA.

4. The total PEEK wedge implant system of claim 3, wherein the wedge implant is manufactured to a desired restoration length necessary for the patient and has an outer anatomical profile that mimics anatomical considerations of a patient's bones that it is being inserted between.

5. The total PEEK wedge implant system of claim 4, wherein the wedge implant has a height ranging from about 10 mm to 50 mm, a width ranging from about 10 mm to 40 mm, and a thickness ranging from about 1 mm to 20 mm and also has an angle ranging from about 2 degrees to 30 degrees.

6. The total PEEK wedge implant system of claim 5, wherein a body of the wedge implant comprises at least two bores each of which are dimensioned to receive a pin, such that a head of the pin is held by the body of the wedge implant and a portion of a shank of the pin extends from the body for receipt in a foot bone of the patient.

7. The total PEEK wedge implant system of claim 6, wherein a first pin bore extends between a top and a first side of the body such that a tip of a pin extends from the first side at an angle thereof and the second pin bore extends between the top and a second side of the body such that the tip of a pin extends from the second side at an angle thereof.

8. The total PEEK wedge implant system of claim 7, wherein both pin bore openings on the top of the body are sized and configured to capture and retain the head of the pin, such that the heads of the pins are countersunk relative to a surface of the top of the body.

9. The total PEEK wedge implant system of claim 8, wherein each of the pair of the pins includes a head with a hexalobe insertion feature.

10. The total PEEK wedge implant system of claim 9, wherein each of the pair of the pins includes a plurality of anti-migration ribs along an exterior shank to prevent the pair of pins from migrating out of the foot bone.

11. The total PEEK wedge implant system of claim 10, wherein the plurality of anti-migration ribs are cut into an exterior of the pair of pins along their length, at predetermined intervals for optimal fixation.

12. The total PEEK wedge implant system of claim 11, wherein the plurality of anti-migration ribs are cut in approximately ¼-½ mm radially, such that surrounding bone enters between the plurality of anti-migration ribs when the pin is inserted.

13. The total PEEK wedge implant system of claim 12, wherein the plurality of anti-migration ribs are spaced apart, such that they have a pitch of approximately 2-3 millimeters to promote boney apposition.

14. The total PEEK wedge implant system of claim 13, wherein the body of the wedge implant comprises an opening that extends from the first side to the second side to receive and hold bone graft material.

15. The total PEEK wedge implant system of claim 14, wherein the opening of the body of the wedge implant is filled with an osteo-integrative lattice.

16. The total PEEK wedge implant system of claim 15, wherein the body of the wedge implant and the osteo-integrative lattice are 3D printed together as one piece to provide a solid construct.

17. The total PEEK wedge implant system of claim 16, wherein the body of the wedge implant comprises features for promoting fusion between two foot bones or foot bone portions, such as teeth, serrations or any other anti-migration features.

18. The total PEEK wedge implant system of claim 17, wherein the body of the wedge implant comprises a threaded bore insertion feature that is provided for engagement with an insertion instrument or drill guide for implantation of the wedge implant.

19. A wedge implant comprising:
a wedge implant made to a desired restoration length necessary for a user and has an outer anatomical profile that mimics anatomical considerations of bones it is being inserted between;
wherein the wedge implant is manufactured of PEEK HA;
wherein the wedge implant is utilized to provide a bi-planar correction; and
further wherein the bi-planar correction includes correction in a transverse plane to bring a toe back into alignment and correction in a sagittal plane to allow for proper gate of a foot.

20. A wedge implant comprising:
a wedge implant comprising an anatomically-shaped body with at least two bores each of with are dimensioned to receive a pin and an opening that extends from the first side to the second side to receive and hold bone graft material;
wherein a first pin bore extends between a top and a first side of the anatomically-shaped body such that a tip of a pin extends from the first side at an angle thereof and the second pin bore extends between the top and a second side such that the tip of a pin extends from the second side at an angle thereof;
wherein the opening of the anatomically-shaped body is filled with an osteo-integrative lattice;
wherein the anatomically-shaped body of the wedge implant comprises features for promoting fusion between two foot bones or foot bone portions, such as teeth, serrations or any other anti-migration features;
wherein the anatomically-shaped body of the wedge implant comprises a threaded bore insertion feature that is provided for engagement with an insertion instrument or drill guide for implantation of the wedge implant;
wherein the wedge implant is manufactured of PEEK HA;
wherein the wedge implant is utilized to provide bi-planar correction; and
further wherein the wedge implant has a height ranging from about 10 mm to 50 mm, a width ranging from about 10 mm to 40 mm, and a thickness ranging from about 1 mm to 20 mm, also having an angle ranging from about 2 degrees to 30 degrees.
